(19) United States
(12) Patent Application Publication
(10) Pub. No.: US 2018/0050980 A1
(43) Pub. Date: Feb. 22, 2018

(54) METHOD FOR PREPARING TETRAMETHYLAMMONIUM FLUORIDE

(71) Applicant: Dow AgroSciences LLC, Indianapolis, IN (US)

(72) Inventors: Douglas C. Bland, Midland, MI (US); Yang Cheng, Midland, MI (US)

(73) Assignee: Dow AgroSciences LLC, Indianapolis, IN (US)

(21) Appl. No.: 15/673,645

(22) Filed: Aug. 10, 2017

Related U.S. Application Data
(60) Provisional application No. 62/376,429, filed on Aug. 18, 2016.

(51) Int. Cl. C07C 209/68; C07C 211/63

(52) U.S. Cl. CPC C07C 209/68; C07C 211/63

(57) ABSTRACT

A method for the preparation of anhydrous, alcohol-free tetramethylammonium fluoride is described. The method includes fluorination of tetramethylammonium chloride with potassium fluoride in an alcohol solvent, removal of insoluble potassium salts, and finally, removal of the alcohol solvent and replacement of it with an aprotic solvent.

This application claims a priority based on provisional application 62/376,429 which was filed in the U.S. Patent and Trademark Office on Aug. 18, 2016. The entire disclosure of which is hereby incorporated by reference.

### FIELD

A method for the preparation of tetramethylammonium fluoride, more particularly, a method for the preparation of anhydrous, alcohol-free tetramethylammonium fluoride is provided.

### BACKGROUND

Anhydrous tetramethylammonium fluoride (TMAF) has been shown to be a useful reagent for the room temperature fluorination of aryl and heteroaryl halides as described in J. Org. Chem. 2015, 80, 12137. However, current methods for preparing TMAF are either costly or involve the tedious handling and drying of the thermally sensitive, solid TMAF.

### SUMMARY

A method for preparing anhydrous, alcohol-free tetramethylammonium fluoride is provided. The method involves (a) mixing together tetramethylammonium chloride (Formula I), potassium fluoride (KF) and an alcohol solvent, (b) isolating tetramethylammonium fluoride (TMAF; Formula II) from the mixture as a solution in the alcohol solvent, (c) adding an aprotic solvent to the mixture containing the tetramethylammonium fluoride as a solution in the alcohol solvent, and (d) removing the alcohol solvent.

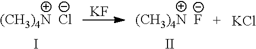

Also described is an anhydrous, alcohol-free composition of tetramethylammonium fluoride in an aprotic solvent that is prepared by the method described herein.

### DETAILED DESCRIPTION

The inventors described herein a novel, convenient, and readily scalable method for the preparation of anhydrous, alcohol-free tetramethylammonium fluoride (TMAF). The method involves: (1) fluorination of tetramethylammonium chloride (TMAC) with a fluoride source, such as potassium fluoride (KF), in an alcohol solvent, (2) removal of insoluble potassium salts (i.e., residual KF and byproduct potassium chloride (KCl)) present in the reaction mixture containing the TMAF by, for example, filtration or centrifugation, and (3) removal of the alcohol solvent by distillation and replacement of it with an aprotic solvent.

The KF used in the method may be obtained from commercial sources and used as received or it may be prepared by drying commercially available KF by any of the drying methods commonly used in the art such as, but not limited to, spray drying, oven drying or fluid bed drying. The particle size of the KF may be reduced to a smaller size by grinding, pulverizing, milling or any other size reduction method commonly used in the art.

While a stoichiometric amount of KF is required to convert TMAC to TMAF, an excess of KF is often employed. The amount of KF used in the described method may range from about 0.5 molar equivalent to about 20 molar equivalents relative to the amount of TMAC used. In some embodiments, the KF:TMAC molar ratio used in the method may be at least about 5:1, at least about 4:1, at least about 3:1, at least about 2.5:1, at least about 2:1, at least about 1.5:1, or at least about 1:1. In some embodiments, the KF:TMAC molar ratio used in the method may range from about 3:1 to about 1:1.

Solvents useful in converting TMAC to TMAF with KF in the described method include alcohols such as, but not limited to, methanol, ethanol, 2-propanol and mixtures thereof. Additional solvents may include mixtures of one or more of the alcohols with one or more aprotic solvents selected from NN-dimethylformamide (DMF), N,N-dimethylacetamide (DMA), sulfolane, cyclic carbonates such as ethylene carbonate and propylene carbonate, and ethers such as, for example, tetrahydrofuran (THF), dioxane, mono- and diethyleneglycol ethers, and mono- and dipropyleneglycol ethers. The amount of TMAC used in the method relative to the solvent include up to about 50 wt % of the total combined weight of TMAC and the solvent. Suitable amounts of TMAC used in the method may be at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, or at least about 50 wt % of the total combined weight of TMAC and the solvent.

In some embodiments, the temperature of the fluorination step in the described method may range from about 10° C. to about 100° C. In some embodiments, the temperature of the fluorination step may be at least about 10° C., at least about 20° C., at least about 30° C., at least about 40° C., at least about 50° C., at least about 60° C., at least about 70° C., at least about 80° C., or at least about 90° C.

In some embodiments sodium fluoride (NaF), lithium fluoride (LiF), or cesium fluoride (CsF) may be used in place of KF to prepare TMAF from TMAC.

The second step in the described method involves removal of the insoluble potassium, sodium, lithium, or cesium salts (i.e., residual KF and byproduct potassium chloride (KCl)) present in the reaction mixture containing the soluble TMAF. The removal of the insoluble salts may be conducted by filtration, centrifugation or by other means known in the art and may provide a visually clear or nearly visually clear solution containing the TMAF. The removed insoluble salts may be washed with one or more portions of the alcohol, to more completely recover the TMAF, and these washings may then be combined with the clear or near clear solution containing the TMAF.

The third step in the described method involves conducting a solvent exchange by removing the alcohol solvent by distillation from the clear solution containing the TMAF and replacing it with an aprotic solvent. Suitable aprotic solvents for use in the solvent exchange may include N,N-dimethylformamide (DMF), N,N-dimethylacetamide (DMA), sulfolane, tetrahydrofuran (THF), 2-methyltetrahydrofuran, cyclopentyl methyl ether (CPME), N-methyl-2-pyrrolidone, dimethyl sulfoxide, cyclic carbonates such as ethylene carbonate and propylene carbonate, dioxane, mono- and diethyleneglycol ethers, mono- and dipropyleneglycol ethers, aromatic solvents, and aliphatic solvents.

Aromatic solvents for use in the solvent exchange may include, for example, solvent naphtha, light aromatics which are sometimes known as Aromatic 100 Fluid; solvent naphtha, heavy aromatics, high flash aromatic naphtha type II, heavy aromatic solvent naphtha, C10 aromatics, which are sometimes known as Aromatic 150 Fluid, A150, and S150 (e.g., Solvesso 150); and solvent naphtha, heavy aromatics, high flash aromatic naphtha type II, heavy aromatic solvent naphtha, C10-13 aromatics, which are sometimes known as Aromatic 200 Fluid, A200, and 5200 (Solvesso 200). Additional aromatic solvents for use in the solvent exchange may include toluene, ethylbenzene and one or more xylenes.

Aliphatic solvents for use in the solvent exchange may include linear, branched and cyclic aliphatic hydrocarbons including, but not limited to, C₆-C₁₅ alkanes such as, for example, n-hexane, cyclohexane, n-heptane, methylcyclohexane and n-octane.

Another embodiment of the third step to remove the alcohol solvent is to distill the methanol/TMAF mixture to dryness yielding a solid TMAF product. This can be accomplished using temperatures between 20-150 degrees C. and vacuum levels between 0.1 and 100 mm of mercury.

In one embodiment, the alcohol solvent is methanol and the aprotic solvent is DMF.

In another embodiment, the alcohol solvent is ethanol and the aprotic solvent is DMF. In another embodiment, the alcohol solvent is 2-propanol and the aprotic solvent is DMF.

In another embodiment, the distillation may be conducted at a vacuum of about 2 mm of mercury to about 200 mm of mercury, a distillation pot temperature ranging from about 10° C. to about 150° C., a distillation vapor temperature ranging from about 10° C. to about 150° C., and by optionally using a distillation column that provides sufficient throughput and separation capacity such as, for example, a 10-tray Oldershaw column, and whereby the distillation column is operated at a reflux ratio of about 10:1 to about 1:1000.

In another embodiment, the distillation may be conducted without a distillation column.

In one embodiment, the distillation may be conducted at a vacuum of less than about 5 mm of mercury, less than about 10 mm of mercury, less than about 20 mm of mercury, less than about 30 mm of mercury, less than about 40 mm of mercury, less than about 50 mm of mercury, less than about 60 mm of mercury, less than about 70 mm of mercury, less than about 80 mm of mercury, less than about 90 mm of mercury, less than about 100 mm of mercury, less than about 125 mm of mercury, less than about 150 mm of mercury, less than about 175 mm of mercury, or less than about 200 mm of mercury.

In one embodiment, the distillation pot temperature may be less than about 10° C., less than about 20° C., less than about 30° C., less than about 40° C., less than about 50° C., less than about 60° C., less than about 70° C., less than about 80° C., less than about 90° C., less than about 100° C., less than about 110° C., less than about 120° C., less than about 130° C., less than about 140° C., or less than about 150° C.

In one embodiment, the distillation vapor temperature may be less than about 10° C., less than about 20° C., less than about 30° C., less than about 40° C., less than about 50° C., less than about 60° C., less than about 70° C., less than about 80° C., less than about 90° C., less than about 100° C., less than about 110° C., less than about 120° C., less than about 130° C., less than about 140° C., or less than about 150° C.

In one embodiment, the distillation may be conducted at a reflux ratio of less than about 10:1, less than about 9:1, less than about 8:1, less than about 7:1, less than about 6:1, less than about 5:1, less than about 4:1, less than about 3:1, less than about 2:1, less than about 1:1, less than about 1:2, less than about 1:3, less than about 1:4, less than about 1:5, less than about 1:6, less than about 1:7, less than about 1:8, less than about 1:9, or less than about 1:10.

The removal of the alcohol solvent and replacement with the aprotic solvent (i.e., the solvent exchange) in the described method may be conducted: 1) in a sequential manner such as, for example, by adding the aprotic solvent to the clear solution containing the TMAF in the alcohol solvent and then removing the alcohol solvent by distillation, and repeating this sequence as needed to completely or nearly completely remove the alcohol solvent, or 2) by continuously adding the aprotic solvent to the solution containing the TMAF in the alcohol solvent while continuously removing the alcohol solvent by distillation.

During the solvent exchange, significant amounts of the aprotic solvent may also be removed during the distillation which may require adding additional amounts of the aprotic solvent during the solvent exchange. Upon completion of the solvent exchange and cooling to ambient temperature, a TMAF-aprotic solvent mixture is formed.

The removal of the alcohol solvent and replacement with the aprotic solvent (i.e., the solvent exchange) in the described method may also be conducted by first removing all or nearly all of the alcohol solvent by distillation or evaporation, optionally under a full or partial vacuum, and then adding the aprotic solvent to the remaining TMAF to form a TMAF-aprotic solvent mixture. Subjecting the resulting TMAF-aprotic solvent mixture to distillation, optionally under vacuum, may then allow for removal of any remaining alcohol solvent by co-distillation with the aprotic solvent to provide the described anhydrous, alcohol-free TMAF-aprotic solvent mixture. The phrase “removing all or nearly all of the alcohol solvent” means removing at least about 80%, at least about 85%, at least about 90%, at least about 92%, at least about 94%, at least about 96%, at least about 97%, at least about 98%, or at least about 99% of the alcohol solvent present in the alcohol-TMAF mixture.

In one embodiment, after the solvent exchange has been completed, the amount of the alcohol solvent remaining in the TMAF-aprotic solvent mixture (i.e., the alcohol-free TMAF-aprotic solvent mixture) is less than about 1000 ppm, less than about 500 ppm, less than about 250 ppm, less than about 200 ppm, less than about 100 ppm, less than about 50 ppm, or less than about 25 ppm by weight compared to the total weight of the TMAF-aprotic solvent mixture.

In another embodiment, after the solvent exchange or solids formation has been completed, the amount of water remaining in the TMAF-aprotic solvent mixture (i.e., the anhydrous or water-free TMAF-aprotic solvent mixture) is less than about 2000 ppm, less than about 1000 ppm, less than about 500 ppm, less than about 250 ppm, less than about 200 ppm, less than about 100 ppm, less than about 50 ppm, or less than about 25 ppm by weight compared to the total weight of the TMAF-aprotic solvent mixture.

Also described is an anhydrous, alcohol-free composition of tetramethylammonium fluoride (TMAF) in an aprotic solvent that is prepared by the method described herein. In one embodiment, the anhydrous, alcohol-free composition of TMAF in the aprotic solvent comprises a mixture of the solid TMAF in the aprotic solvent. The anhydrous, alcohol-free composition of TMAF in the aprotic solvent may include no more than about 50 wt % TMAF, no more than about 40 wt % TMAF, no more than about 30 wt % TMAF, no more than about 25 wt % TMAF, no more than about 20 wt % TMAF, no more than about 15 wt % TMAF, no more than about 10 wt % TMAF, no more than about 7 wt % TMAF, or no more than about 5 wt % TMAF.

The following examples are presented to illustrate the methods and compositions described herein.

### EXAMPLES

### Example 1: Preparation of Me₄NF (TMAF) from Me₄NCl (TMAC) and KF in Methanol

Into a 1 L, 3-necked flask equipped with a stirring bar and a thermometer, Me₄NCl (164.4 g, 1.5 mol, technical grade from SACHEM) and MeOH (650 mL, HPLC grade from Fisher) were charged. The mixture was stirred for 15 minutes until all Me₄NCl was dissolved in the MeOH. The temperature dropped from 22 C to 9° C. and KF (174.3 g, 3 mol, spray-dried material) was added in portions. The reaction was covered under nitrogen and the mixture was stirred at room temperature for 5 hours. The KCl/KF salt suspension that formed was filtered to provide a filtrate and the filtered solids that were washed with MeOH (3×100 mL). The combined filtrate and washings (750.5 g, TMAF/MeOH solution) were stored in a 1 L amber bottle. The water content was analyzed by using a Karl Fischer Coulometer and the TMAF/MeOH solution was found to contain 2127 ppm of water. After drying at 55° C. under a flow of nitrogen for 18 hours, 190 g of the dried, filtered KCl/KF salts were obtained.

### Example 2: Preparation of Me₄NF (TMAF) from Me₄NCl (TMAC) and KF in 2-propanol

Into a 1 L, 3-necked flask equipped with a stirring bar and a condenser, Me₄NCl (82.2 g, 0.75 mol, technical grade from SACHEM) and 2-propanol (650 mL, HPLC grade from Fisher) were charged. The mixture was heated and stirred until all Me₄NCl was dissolved in the 2-propanol. KF (87.15 g, 1.5 mol, spray-dried material) was added in portions and the resulting mixture was heated (about 83° C.) to reflux under nitrogen and stirred for 8 hours. The mixture was then cooled to room temperature and stirred at room temperature overnight. The KCl/KF salt suspension that formed was filtered to provide a filtrate and the filtered solids that were washed with 2-propanol (3×50 mL). The combined filtrate and washings (587.4 g, TMAF/2-propanol) were stored in a 1 L amber bottle. The water content was analyzed by using a Karl Fischer Coulometer and the TMAF/2-propanol solution was found to contain 2676 ppm of water. After drying at 55° C. under a flow of nitrogen for 18 hours, 114.1 g of the dried, filtered KCl/KF salts were obtained.

### Example 3: Preparation of Methanol Free Me₄NF (TMAF) by Vacuum Distillation

The TMAF/MeOH solution (prepared as described in Example 1, 150 g; containing ca. 0.30 mol of TMAF) and DMF (1 L, 936 g, Fisher Spectraanalyzed® grade) were charged into a distillation pot (2 L, 4-necked flask) fitted with a 10-tray Oldershaw distillation column. The distillation was held at full vacuum (4.3 mm Hg) for 1 hr, and the pot temperature dropped to 12° C. The pot temperature was then increased to 48° C. while holding 2.7 mm Hg vacuum resulting in a condenser vapor temperature of 22° C. The reflux ratio was set at 3:1, and clear liquid (65.3 g) was collected in the receiving flask over 13.5 hrs. A sample was taken from the pot for methanol content analysis. ¹H NMR analysis indicated that the mole ratio of MeOH/TMAF was 1.22:1.

The distillation was continued under the conditions of a pot temperature of about 50° C., a vacuum of about 2.5 mm Hg, a condenser vapor temperature of about 27° C. and at a reflux ratio of about 3:1. The distillation was stopped after operating for 13.5 hours at these conditions resulting in an additional 80.5 g of clear distillate liquid. ¹H NMR analysis of the sample from the pot showed the mole ratio of MeOH/TMAF=0.81:1. The distillation was further continued at a pot temperature of about 72° C., a vacuum of about 36.5 mm Hg, a condenser vapor temperature of about 65° C. and at a reflux ratio of about 3:1 for another 10.25 hrs. More clear liquid (271.9 g) was collected in the receiving flask. A sample was taken from the pot for methanol analysis. Based on ¹H NMR, the mole ratio of MeOH/TMAF was 0.15:1.

The distillation was continued at a pot temperature of about 72° C., a vacuum of about 36.5 mm Hg, and a condenser vapor temperature of about 65° C. for another 5.5 hr. The reflux ratio was adjusted from about 3:1 to about 3:8 to increase the collection rate. About 195 g of clear liquid was collected in the receiving flask. A sample was taken from the pot for methanol analysis. ¹H NMR showed the mole ratio of MeOH/TMAF was 0.06:1, and GC analysis showed the methanol content was 1696 ppm.

Overall at this time, about 612 g of distillate was collected, and the volume in the pot was becoming low. Fresh, anhydrous DMF (534 g) was added to the pot for further distillation. The distillation conditions were pot temperature (73° C.), vacuum (36.8 mm Hg), condenser vapor temp=66° C. and reflux ratio=3:1. The distillation was stopped after another 11.5 hr. About 133.6 g of clear liquid was collected in the receiving flask, and a sample was taken from the pot for methanol content analysis. The MeOH peak was not able to be quantified by ¹H NMR analysis. GC analysis showed MeOH content was 132 ppm.

The distillation was continued under the conditions of pot temperature (72° C.), vacuum (36.2 mm Hg), and condenser vapor temperature (66° C.). The reflux ratio was started at 15:5 and adjusted to 15:10, 15:15 to increase the collection rate. DMF (124.7 g) was collected in the receiving flask during another 14 hours of distillation. By then, GC analysis showed the MeOH content in the pot was 45 ppm. The mixture was cooled to room temperature. The TMAF/DMF (377.8 g) mixture was transferred into a bottle and stored in a glove box. Analysis of the TMAF/DMF mixture by ion chromatography indicated the presence of 5.98 weight % TMAF. From the entire distillation run (68 hours), about 872 g of clear liquid (mostly DMF) was collected in the receiving flask.

### Example 4: Preparation of 2-Propanol Free Me₄NF (TMAF) by Vacuum Distillation

The preparation of 2-propanol free TMAF was carried out in the same distillation equipment setup as used in Example 3. TMAF/2-propanol solution (prepared as described in Example 2, 150 g) and DMF (1 L, 936 g, Fisher Spectraanalyzed® grade) were charged into the pot (2 L, 4-necked flask). The distillation was held at full vacuum (7.5 mm Hg) for 1 hr, and the pot temperature dropped to 17° C. Then the pot was heated to 70° C. with 60 mm Hg vacuum and overheads were collected using a reflux ratio of 1:1. When the liquid drip rate on the condenser slowed down relative to the initial rate, the pot temperature was increased to 81° C. The vacuum was kept at 60 mm Hg and the vapor temperature on the condenser was 61° C. The distillation was stopped when the liquid drip rate on the condenser slowed again. Approximately 169.2 g of a clear distillate liquid was collected. A sample was taken from the pot for 2-propanol content analysis. ¹H NMR analysis indicated that the mole ratio of 2-propanol/TMAF=1.1:1.

The distillation was continued under the conditions of a pot temperature of about 68° C., a vacuum of about 26 mm Hg, a condenser vapor temperature of about 57° C. and a reflux ratio of about 3:1. A clear liquid (131.2 g) was collected in the receiving flask. It was also found that a clear liquid (32.3 g) was collected in the dry ice trap. ¹H NMR analysis of the sample from the pot showing the mole ratio of 2-propanol/TMAF=0.16:1.

The distillation was further continued at pot temperature of about 74° C., a vacuum of about 26.9 mm Hg, a condenser vapor temperature of about 58° C. and reflux ratio of about 3:1. A clear liquid (351.9 g) was collected in the receiving flask. A sample was taken from the pot for 2-propanol analysis. Based on ¹H NMR, the mole ratio of 2-propanol/TMAF was less than 0.01:1.

Fresh anhydrous DMF (256.3 g) was added to the pot for further distillation. The distillation conditions were pot temperature of about 70° C., a vacuum of about 26.2 mm Hg, a condenser vapor temp of about 58.7° C. and a reflux ratio of about 3:1. A clear liquid (267.1) g was collected in receiving flask, and a sample from the pot was taken for methanol content analysis. GC analysis showed the 2-propanol content was 47 ppm.

The mixture was cooled to room temperature. The TMAF/DMF mixture (329.1 g) was transferred to a bottle and stored in a glove box. From the entire experiment, the clear liquid (919 g, most was DMF) was collected in the receiving flask. The amount of material collected in the dry ice trap was 36.7 g.

### Example 5: Preparation of methanol-free MealVF (TMAF) in 2-methyltetrahydrofuran (2-Me-THF) by vacuum distillation using a partial condenser

The TMAF/MeOH solution will be prepared as described in Example 1. The clear solution containing the TMAF in MeOH will be charged to the distillation pot and will be subjected to vacuum distillation. The MeOH will be distilled overhead and methyl-THF will be added to the distillation pot during the course of the MeOH removal and/or prior to beginning methanol removal. The MeOH will be removed to similar levels as in Example 3 (i.e., <100 ppm MeOH). The final mixture containing the MeOH-free TMAF in 2-Me-THF will be transferred to a bottle and stored in a glove box.

### Example 6: Preparation of Methanol-Free Me₄NF (TMAF) in Tetrahydrofuran by Vacuum Distillation Using a Partial Condenser

The TMAF/MeOH solution will be prepared as described in Example 1. The clear solution containing the TMAF in MeOH will be charged to the distillation pot and will be subjected to vacuum distillation. The MeOH will be distilled overhead and THF will be added to the distillation pot during the course of the MeOH removal and/or prior to beginning methanol removal. The MeOH will be removed by azeotropic distillation after addition of the THF. The MeOH will be removed to similar levels as in Example 3 (i.e., <100 ppm MeOH). The final mixture containing the MeOH-free TMAF in THF will be transferred to a bottle and stored in a glove box.

The compositions and methods of the claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative composition materials and method steps disclosed herein are specifically described, other combinations of the composition materials and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

What is claimed is:

1. A method for preparing anhydrous, alcohol-free tetramethylammonium fluoride which comprises:
a) mixing together tetramethylammonium chloride, potassium fluoride and an alcohol solvent;
b) isolating tetramethylammonium fluoride from the mixture as a solution in the alcohol solvent;
c) adding an aprotic solvent to the mixture containing the tetramethylammonium fluoride as a solution in the alcohol solvent; and
d) removing the alcohol solvent.

2. The method of claim 1 in which the alcohol solvent is selected from the group including methanol, ethanol, and 2-propanol.

3. The method of claim 1 in which the tetramethylammonium fluoride as a solution in the alcohol solvent is isolated by removing insoluble potassium salts from the first mixture.

4. The method of claim 3 in which the removing insoluble potassium salts is conducted by filtration or centrifugation.

5. The method of claim 1 in which the aprotic solvent may be selected from the group including N,N-dimethylformamide (DMF), N,N-dimethylacetamide (DMA), sulfolane, 2-methyl tetrahydrofuran, N-methyl-2-pyrrolidone, dimethyl sulfoxide, tetrahydrofuran, cyclopentyl methyl ether, ethylene carbonate, propylene carbonate, dioxane, mono- and diethyleneglycol ethers, and mono- and dipropyleneglycol ethers.

6. The method of claim 1 in which the alcohol solvent is removed by distillation.

7. The method of claim 6 in which the distillation is a vacuum distillation.

8. An anhydrous, alcohol-free composition of tetramethylammonium fluoride in an aprotic solvent prepared by the method of claim 1.

9. The anhydrous, alcohol-free composition of claim 8, wherein the tetramethylammonium fluoride is a mixture of tetramethylammonium fluoride in the aprotic solvent.

10. The method of claim 1, wherein all or nearly all of the alcohol solvent is removed from the mixture prior to adding the aprotic solvent.

11. The method of claim 1 wherein the mixing together tetramethylammonium chloride, potassium fluoride and an alcohol solvent further comprises an aprotic solvent.

12. The method of claims 1-12 wherein sodium, lithium, or cesium fluoride is used in place of potassium fluoride.
